(19) United States
(12) Patent Application Publication
(10) Pub. No.: US 2009/0203947 A1
(43) Pub. Date: Aug. 13, 2009

(54) Method for Oligomerization of Ethylene and Reactor System therefor with Cooling Device

(76) Inventors: Richard Schneider, Uffing (DE); Peter M. Fritz, Unterhaching (DE); Sebastian Muschelknautz, Munich (DE); Heinz Bölt, Wolfratshausen (DE); Talal Ali, Riyadh (SA); Fuad Mosa, Riyadh (SA)

Correspondence Address: KRAMER LEVIN NAFTALIS & FRANKEL LLP;INTELLECTUAL PROPERTY DEPARTMENT, 1177 AVENUE OF THE AMERICAS, NEW YORK, NY, 10036, US

(21) Appl. No.: 12/083,786

(22) PCT Filed: Sep. 5, 2006
(86) PCT No.: PCT/EP2006/008622

§ 371 Date: Feb. 9, 2009

(30) Foreign Application Priority Data
Oct. 20, 2005 (EP) 05022864.2

(51) Int. Cl. C07C 7/09; B01J 19/24; C07C 2/08

(52) U.S. Cl. 585/519; 422/198

(57) ABSTRACT

The present invention relates to a method for oligomerisation of ethylene to form linear alpha-olefins in an oligomerisation reactor in the presence of solvent and catalyst, wherein a reaction product containing unreacted ethylene and light linear alpha-olefins is discharged from the oligomerisation reactor and passed to at least one first direct cooling device for separating the reaction product into an ethylene rich gaseous fraction and a light linear alpha-olefins rich liquid fraction, wherein at least a portion of the light linear alpha-olefins rich liquid fraction is passed to a second cooling device for lowering the temperature thereof and is subsequently re-introduced into the first direct cooling device; and to a reactor system therefore.

The present invention relates to a method for oligomerisation of ethylene to form linear alpha-olefins in an oligomerisation reactor in the presence of solvent and catalyst, wherein a reaction product containing unreacted ethylene and light linear alpha-olefins is discharged from the reactor and passed to at least one first direct cooling device for separating the reaction product into an ethylene rich gaseous fraction and a light linear alpha-olefins richt liquid fraction, and to a reactor system therefore.

Methods for oligomerisation of ethylene are widely known in the art. For example, DE 43 38 414 C1 discloses a process for the preparation of linear alpha-olefins by oligomerisation of ethylene, wherein oligomerisation takes place in the presence of an organic solvent and a homogenous liquid catalyst. Usually, a catalyst is utilized in that process comprising a zirconium component and an organoaluminum component which acts as a co-catalyst. The oligomerisation may be carried out in that starting material comprising monomer, catalyst, co-catalyst and solvent is transferred to a reactor equipment. After conversion in the reactor equipment, product material comprising oligomers, non-reacted monomer, catalyst, co-catalyst and solvent may be discharged from the reactor equipment and can be further processed. According to DE 43 38 414 C1 a reaction product containing unreacted ethylene and light alpha-olefins may be discharged and transferred to a heat exchanger where the mixture is cooled to a specific temperature, for example 35° C. The liquid fraction obtained by cooling (containing substantially light alpha-olefins) is re-introduced into the reactor. The gaseous fraction obtained (substantially containing unreacted ethylene) is transferred to another heat exchanger where the temperature is further decreased, for example to a temperature of about 5° C. In the second heat exchanger unreacted ethylene is substantially separated from remaining light alpha-olefins. The separated alpha-olefins may then be further processed, for example in a rectification column.

Thus, in prior art methods for oligomerisation of ethylene, relatively small amounts of light linear alpha-olefins are generally separated from a relatively large gaseous ethylene stream by partial condensation in several heat exchangers. As the heat-transfer coefficients of the gaseous components are relatively small, the heat exchangers utilized are relatively large and must be supported in complicated constructions, such as steel constructions.

It is an object of the present invention to provide a method for oligomerisation of ethylene which overcomes the drawbacks of the prior art. Especially a method shall be provided which may utilize smaller heat exchangers which do not have to be incorporated in complicated constructions.

Additionally, it is an object of the present invention to provide a reactor system for carrying out the inventive method.

The first object is achieved in that at least a portion of the light linear alpha-olefins rich liquid fraction is passed to a second cooling device for lowering the temperature thereof and is subsequently re-introduced into the direct cooling device.

Preferably, re-introduction is at the top of the direct cooling device.

In addition it is also preferred that unreacted ethylene is recycled into the oligomerisation reactor or further processed.

In a preferred embodiment another portion of the light alpha-olefins rich liquid fraction is transferred to a separation device.

Preferably, the separation device is a rectification column.

In addition it is also preferred, that the first direct cooling device contains random packing, structured packing and/or trays.

In one aspect to the light alpha-olefins rich liquid fraction being re-introduced into the first direct cooling device an additional solvent is added.

In a preferred embodiment the additional solvent is a heavier alpha-olefins fraction, preferably also obtained in the oligomersation method.

In one aspect, the light alpha-olefins are C₄-C₈-olefins and/or heavy alpha-olefins are C₁₀-C₁₈-olefins

In addition it is also preferred that the light alpha-olefins rich fraction is cooled in the second cooling device to a temperature of about 3° C. The mixture of ethylene and light linear alpha-olefins is cooled in the first direct cooling device preferably to a temperature of about 8° C.

The second object is achieved by a reactor system for oligomerisation of ethylene to form linear alpha-olefins, preferably utilizing an inventive method, comprising an oligomerisation reactor and a separation unit connected thereto for separating unreacted ethylene from light alpha-olefins, the separation unit comprising at least one direct cooling device having a loop-line for light alpha-olefins passing through a second cooling device.

According to the present invention, the heat is advantageously removed from liquid stream (light linear alpha olefins) instead of a gaseous stream as done so far in the prior art. In fact, the heat is removed from the loop cycling light liquid alpha-olefins. Thereby, substantially higher heat-transfer coefficients may be realized. Thus, the heat exchangers and cooling devices utilized in the method of the present invention may be designed substantially smaller and do not need to be integrated in complicated constructions. The method of the present invention additionally provides an increased separation efficiency, since in the direct cooling device more than one theoretical separation stage can be applied.

Additional features and advantages of the inventive method and reactor system are further illustrated with reference to the accompanying drawing, wherein

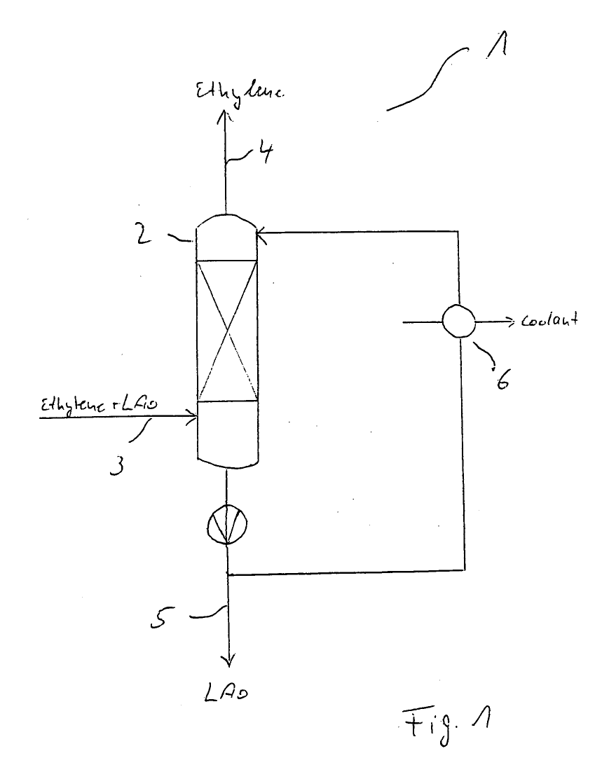

FIG. 1 illustrates a schematic diagram of a separation unit which form part of the inventive reactor system.

FIG. 1 illustrates a separation unit 1 comprising a first direct cooling device 2. From an oligomerisation reactor (not shown) unreacted ethylene and light linear alpha-olefins are transferred into the first direct cooling device 2 via line 3 directly or indirectly. In the first direct cooling device 2, the mixture of unreacted ethylene and light linear alpha-olefins is cooled to a specific temperature, for example 8° C., so that the reaction product is separated into an ethylene rich gaseous fraction and an light alpha-olefins rich liquid fraction. The ethylene rich fraction may be discharged from the first direct cooling device 2 via line 4 and may be re-introduced into the oligomerisation reactor. The light alpha-olefins rich liquid fraction may be also discharged from the first direct cooling device 2 via line 5. At least a portion of that fraction may, however, be passed to a second cooling device 6, preferably utilizing an adequate coolant, where the light alpha-olefin rich liquid fraction is further cooled, for example to a temperature of about 3° C. The thus cooled light alpha-olefins rich liquid fraction is then re-introduced into the first direct cooling device 2, preferably at the top thereof. Thus, the re-introduced light alpha-olefins fraction may be utilized as washing agent to reduce further the amount of linear alpha-olefins present in the gaseous stream of unreacted ethylene.

As the heat of the reaction products is removed according to the present invention from a liquid stream instead of a gas stream, the sizes of involved heat exchangers or cooling device may be significantly reduced. This may result in cost and energy savings.

Another portion of the light alpha-olefins rich fraction which is not cooled and re-introduced into the first direct cooling device 2, may be transferred to a separation device (not shown) such as a rectification column to fractionate the linear alpha-olefins obtained.

The features disclosed in the foregoing description, in the claims and in the drawing may, both separately and in any combination thereof, be material for realizing the invention in diverse forms thereof.

1. A method for separating linear alpha-olefins formed by the oligomerization of an ethylene feed in the presence of a solvent and a catalyst, from a oligomerization reaction product containing unreacted ethylene and light linear alpha-olefins, comprising introducing the oligomerization reaction product into direct cooling device which separates the reaction product into an ethylene rich gaseous fraction and a light linear alpha-olefins rich liquid fraction, wherein that at least a portion of the light linear alpha-olefins rich liquid fraction obtained is passed to a second cooling device which reduces the temperature of the light linear alpha-olefins rich liquid faction and then re-introduced into the direct cooling device.

2. The method according to claim 1, wherein re-introduction of the light linear alpha-olefins rich liquid fraction is at the top of the direct cooling device.

3. The method according to claim 2, wherein unreacted ethylene is recycled into the ethylene feed.

4. The method according to claim 2, wherein a portion of the light alpha-olefins rich liquid fraction is transferred to a separation device.

5. The method according to claim 4, wherein the separation device is a rectification column.

6. The method according to claim 1, wherein the direct cooling device contains random packing, structured packing and/or trays.

7. The method according to claim 1, wherein the light alpha-olefins rich liquid fraction is re-introduced into the direct cooling device with added solvent.

8. The method according to claim 7, wherein the added solvent comprises a heavier alpha-olefins fraction.

9. The method according to claim 8, wherein light alpha-olefins rich liquid fraction comprises C₄-C₈-olefins and/or the heavier alpha-olefins fraction comprises C₁₀-C₁₈-olefins.

10. The method according to claim 9, wherein the light alpha-olefins rich fraction is cooled in the second cooling device to a temperature of about 3° C.

11. A reactor system for oligomerization of ethylene to form linear alpha-olefins, comprising an oligomerization reactor which produces a reaction product comprised of light linear alpha-olefins and unreacted ethylene, wherein the reaction product is introduced into a direct cooling device for a separation into a light linear alpha-olefins rich liquid fraction and an ethylene rich gas fraction according to claim 1.

12. The method according to claim 2, wherein the direct cooling device contains random packing, structured packing and/or trays.

13. The method according to claim 6, wherein the light alpha-olefins rich liquid fraction is re-introduced into the direct cooling device with added solvent.

14. The method according to claim 1, wherein the light alpha-olefins rich fraction is cooled in the second cooling device to a temperature of about 3° C.

15. The method according to claim 6, wherein the light alpha-olefins rich fraction is cooled in the second cooling device to a temperature of about 3° C.

16. A reactor system for oligomerization of ethylene to form linear alpha-olefins, comprising an oligomerization reactor which produces a reaction product comprised of light linear alpha-olefins and unreacted ethylene, wherein the reaction product is introduced into a direct cooling device for a separation into a light linear alpha-olefins rich liquid fraction and an ethylene rich gas fraction according to claim 6.

17. A reactor system for oligomerization of ethylene to form linear alpha-olefins, comprising an oligomerization reactor which produces a reaction product comprised of light linear alpha-olefins and unreacted ethylene, wherein the reaction product is introduced into a direct cooling device for a separation into a light linear alpha-olefins rich liquid fraction and an ethylene rich gas fraction according to claim 8.
